(19) United States
(12) Patent Application Publication
(10) Pub. No.: US 2014/0155829 A1
(43) Pub. Date: Jun. 5, 2014

(54) DISPOSABLE AMBULATORY INFUSION PUMP HAVING TELESCOPIC HOUSING

(71) Applicant: Seik Oh, Trabuco Canyon, CA (US)

(72) Inventor: Seik Oh, Trabuco Canyon, CA (US)

(21) Appl. No.: 13/691,346

(22) Filed: Nov. 30, 2012

(51) Int. Cl. A61M 5/142

(52) U.S. Cl. CPC A61M 5/14244
USPC 604/131

(57) ABSTRACT

A disposable ambulatory infuser apparatus provides an elastomeric balloon within a telescopic housing. One end of the balloon may be attached to an upper housing portion and the other end of the balloon may be attached to a bottom housing portion. As the balloon is charged with a drug, it may expand primarily radially first and then axially. The balloon may be configured to have the majority of displacement occur in the axial direction, which enables the two housing portions to expand away from each other and shrink toward one another in the axial direction as the balloon is filled with the drug and the drug is subsequently infused in a patient. The balloon may expand axially naturally within the housing to eliminate possible pressure change due to constraints by the housing or clamps, which enables more constant flow rate of the drug being dispensed.

### BACKGROUND OF THE INVENTION

The present invention generally relates medical devices and more particularly, to a disposable ambulatory infusion pump.

Nonelectric disposable infusion pumps have been in clinical use for more than twenty years. Today, disposable infusion pumps are extensively used in hospitals and home care settings to deliver therapies such as chemotherapy, antimicrobials, analgesia, and anesthesia, as well as for postoperative pain management. Patients prefer disposable pumps to electric pumps mainly because of the disposable pump's small size, simplicity in use, and disposability. The small size of disposable pumps is particularly attractive for patients receiving long-term infusions as these pumps can be used with minimal restrictions to routine daily life. Disposable pumps may allow greater freedom of movement than electric devices.

In elastomeric infusion pumps, the pressure is typically generated by the contraction of an inflated elastomeric balloon. The balloon is typically a drug container/dispenser. Among many different disposable ambulatory pumps, the elastomeric pump offers some of the smallest sizes and lightest weights.

The elastomeric balloon is normally protected by a hard shell. A soft outer shell, which is usually made of another layer of elastomer or thin- flexible plastics were introduced to reduce the size because the soft shell shrinks as drug infuses. However, it may offer insufficient protection against external forces or sharp punctures. The soft-shell elastomeric pumps usually employ multiple layer membranes to prevent potential failure of the reservoir by an unexpected external force. Because a multilayer membrane has a strong resistance while filling, the user usually needs to purchase a separate filling apparatus. Also, soft-shell type elastomeric pumps usually have a spherical shape when fully inflated, which causes discomfort to the patient.

In recent years, demand for large volume devices has increased as pain management requires longer term therapies. Currently available balloon type disposable pumps include volumes fixed to certain nominal volumes such as 60 ml, 100 ml, 250 ml and 600 ml.

Some previous elastomeric balloon based designs use the elastomeric balloon as a pressure source. As inflation progresses, the balloon naturally may inflate radially until it is restricted by touching the housing or by inflating to the fixed length of the balloon. These restrictions may change the boundary condition of the inflation. The balloon may develop inconsistent areas of structural integrity with some areas being over subjected to stress and strain. In some instances, the balloon may burst as a result of compromised structural integrity. In other instances the balloon may develop a change in pressure profile and consequently causes inconsistent flow rate of dispensed drugs.

### SUMMARY OF THE INVENTION

In one aspect of the present invention, an infusion pump system comprises a first housing portion including a first end cap on a sealed end of the first housing portion. A second housing portion may be disposed to slide axially within the first housing portion. The second housing portion includes a second end cap on a sealed end of the second housing portion. The sealed end of the second housing portion may be external of the first housing portion. An elastomeric balloon may be coupled to an inlet of the first end cap and coupled to the second end cap. The balloon may be positioned within the first and second housing portions disposed to telescope the first housing portion axially over the second housing portion when the balloon is infused with a fluid.

In another aspect of the present invention, an infusion pump comprises a first cylindrical housing including an inner diameter. A second cylindrical housing may include an outer diameter that is less than the inner diameter of the first cylindrical housing. The second cylindrical housing may be disposed to slide freely within the first cylindrical housing. A first end cap on the first cylindrical housing may include an inlet disposed to provide fluid flow into or out of the first cylindrical housing. The second end cap on the second cylindrical housing may be external of the first cylindrical housing.

These and other features, aspects and advantages of the present invention will become better understood with reference to the following drawings, description and claims.

### BRIEF DESCRIPTION OF THE DRAWINGS

FIG. 1 is a perspective view of an infusion pump system in a default un-inflated state according to one embodiment;

FIG. 2 is a perspective side view of the infusion pump system of FIG. 1 in an inflated state;

FIG. 3 is a cross-sectional side view of infusion pump of FIG. 1;

FIG. 3A is an enlarged view of the circle “3A” in FIG. 3;

FIG. 3B is an enlarged view of the circle “3B” in FIG. 3; and

FIG. 4 is an exploded perspective view of the infusion pump system of FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, embodiments of the present invention generally provide an infusion pump system that expands with the natural mode of the stretch of elastomeric balloon in opposing axial directions. In one aspect, an elastomeric balloon in the infusion pump system may be disposed to expand primarily radially without contacting inner surfaces of surrounding housing and then expand axially encouraging ends of the housing to telescope away from each other providing a more constant pressure in the system. A benefit may be seen in both the structural longevity of the balloon and in the delivery of a drug from the pump may be maintained during deflation of the balloon (for example, during infusion of a drug into a patient).

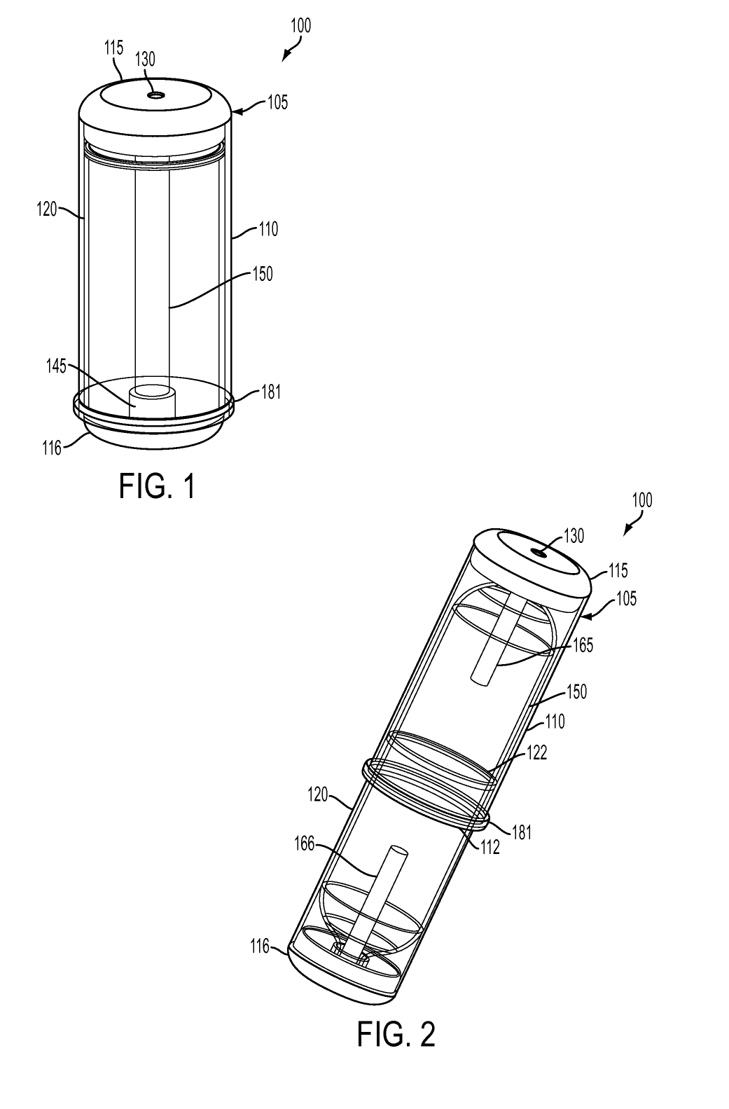

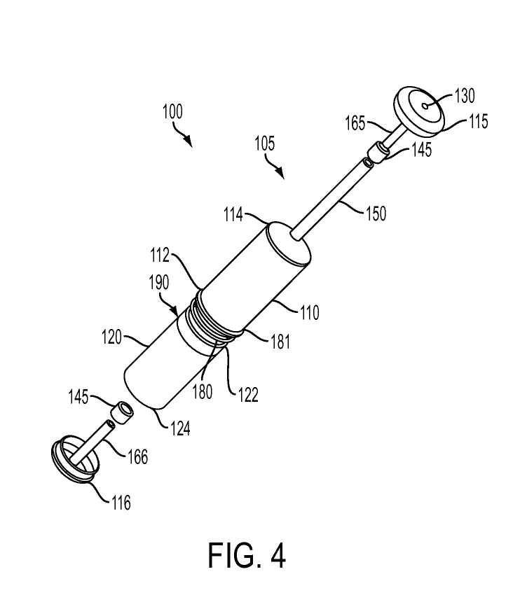

Referring to FIGS. 1-4, an infusion pump system 100 is shown according to an exemplary embodiment of the present invention. FIG. 1 shows a default un-inflated state of the infusion pump system 100. FIG. 2 shows the infusion pump system 100 telescoped into an expanded or inflated state. In an exemplary embodiment, the infusion pump system 100 may include an infusion pump 105 and an elastomeric balloon 150. The infusion pump 105 may be configured to receive and subsequently provide a fluid from its interior. The fluid may be for example, a drug being administered to a patient.

In an exemplary embodiment, the infusion pump 105 may be configured to receive and dispense fluid under a certain pressure and flow rate. For example, the infusion pump 105 may include two cylindrical housing portions 110 and 120. The material of the two cylindrical housing portions 110 and 120 may include thermoplastics like polycarbonate or polyester which can provide structural integrity (protecting the infusion pump interior from external forces) and a view of the contents within the infusion pump 105. In an exemplary embodiment, the housing portion 120 may be disposed to slide axially within the housing portion 110. The housing portion 120 may include an outer diameter 175 that is less than an inner diameter 170 of the housing 110. An open end 112 may be distal from a sealed 114 of the housing portion 110. An open end 122 may be distal from a sealed end 124 of the housing portion 120.

In the default un-inflated state, the open end 122 of the housing portion 120 may fit within the open end 112 of the housing portion 110. The open end 122 may be positioned proximate the sealed end 114. The open end 112 may be proximate the sealed end 124.

When coupled together, the housing portions 110 and 120 may form an enclosed cylindrical body. The housing portion 110 may float over the housing portion 120 with a clearance so that the sealed ends 114 and 124 may telescope simultaneously away from one another when the infusion pump 105 is being filled with fluid. In some embodiments, the housing portion 110 overlaps some of the housing portion 120 when the infusion pump 105 is in the inflated state. Snap bumps 190 and 192 are installed on the outside wall of the housing 120 near the open end 122, and on the inside wall of the housing 110 near the open end 112, respectively, in order to stop the excessive sliding and provide a sufficient overlap between the two housings which can be caused by unexpected over-filling.

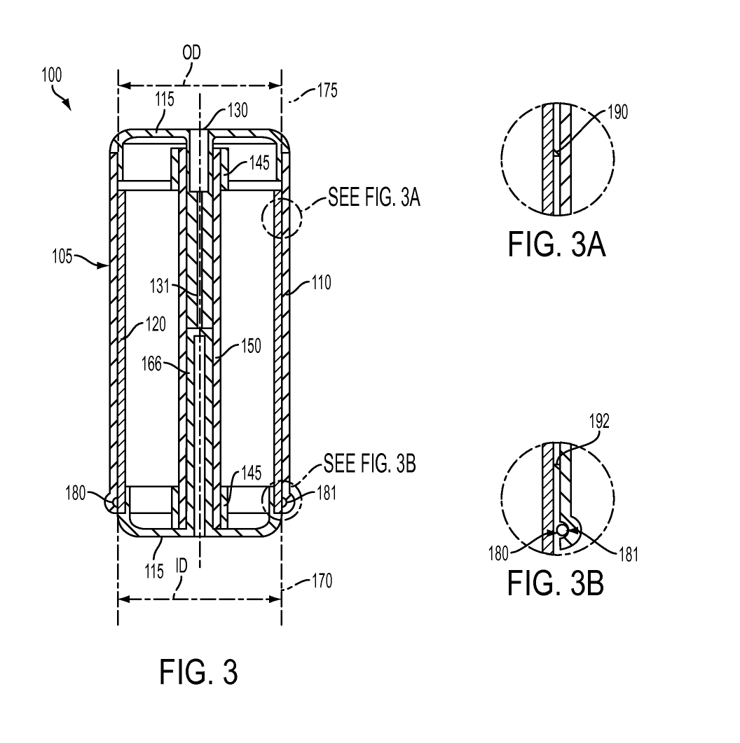

In some embodiments, the housing portion 110 may include a sealing ring 180, for example, an O-ring on a circular groove 181 fabricated to provide a seat for the O-ring adjacent to the open end 112. The sealing ring 180 may provide a slight interference between the housing portions 110 and 120 by supporting the circumferential clearance between the housing portion 110 and the housing portion 120. Thus, the sealing ring 180 may provide stability between the two housing portions 110 and 120 and may prevent wiggling or sticking due to too loose or too tight a clearance. Also, the sealing ring 180 may provide a splash-proof barrier when the balloon 150 leaks. A snap bump 190 may be present on the exterior of the housing portion 120 (FIG. 3A) and a snap bump 192 (FIG. 3B) may be present on the housing portion 110. The snap bumps 190 and 192 may be disposed to limit the sliding movement of the housing portions 110 and 120 preventing for example, separation of the housing portions 110 and 120 from one another due to over inflation of the elastomeric balloon 150.

Both the housing portion 110 and the housing portion 120 may include end caps 115 and 116 respectively sealing the sealed ends 114 and 124 of the infusion pump 105. The end cap 115 may be sized to seal the housing portion 110 which may have a different inner diameter than the housing portion 120. The end cap 116 may accordingly be sized to seal the housing portion 120.

The end cap 115 may include an inlet wall 130 disposed to provide fluid access between the interior and exterior of the infusion pump 105. A post 165 may be coupled to the inlet wall 130. The post 165 may project inward from the end cap 115 into the interior of the housing portion 110. The post 165 may include a hollow inner diameter 131 disposed to carry fluid from the inlet wall 130 to the balloon 150. The post 165 may also include an outer diameter adapted to receive the balloon 150. In operation, fluid may enter the end cap 115 through the inlet wall 130 and may flow through the inner diameter 131 and out of the post 165 filling the balloon 150.

The end cap 116 may be closed. A post 166 may project from the end cap 116 into the interior of the infusion pump 105. An end of the balloon 150 may be fitted around the post 166. The distal end of the post 166 may be sealed preventing fluid from entering into the post. A band clamp 145 may be attached to the base of the post 165 and post 166. When the infusion pump 105 is un-inflated, ends of the posts 165 and 166 may abut one another or be close to contact. The sealed distal end of the post 166 may occupy space within the balloon 150 forcing any residual fluid out through the post 165 deflation of the balloon 150 (e.g. during infusion of the fluid into the patient).

The elastomeric balloon 150 may be disposed to inflate as it receives fluid introduced through the post 165. In an exemplary embodiment, the balloon 150 may be configured to avoid contact with the inner diameter 170 of the housing portion 110 and the inner diameter of the housing portion 120 when the balloon 150 is fully inflated with the fluid. The balloon 150 may be pre-stretched axially between the two end caps 115 and 116 when the infusion pump 105 is in the default un-inflated state. For example, one end of the balloon 150 may be coupled to the end cap 115 and an axial tension may be applied to the balloon as it is coupled to the opposing end cap 116. The band clamp 145 on each of the end caps 115 and 116 may hold the ends of the balloon 150 in place converting the balloon into a cylindrical shaped closed container. The post 165 may be inserted into the one end of the balloon 150 so that fluid from the inlet walls 130 may fill the balloon 150, and the post 166 may be inserted into the other end of the balloon 150 to plug the end and take the empty space of the balloon. The distance between opposing band clamps 145 may be set to be slightly longer than the length of the balloon 150 so that the balloon 150 is stretched, for example 15%-30% more than its natural length.

If the balloon 150 is assumed to be a both ends closed thick wall cylinder, which is subjected to internal pressure, stress occurs in both the circumferential (radial) and longitudinal (axial) directions. Assuming that the elastomer material obeys Hooke's Law, circumferential stress (σ_{θ}) is twice as large as longitudinal stress (σ_{z});

σ_{θ}=2σ_{z}. (1)

For linear elastic material, stress is proportional to the product of elastic modulus, E and strain, ε. Stress increases as strain increases. Also, in elastomeric materials, elastic modulus increases as strain increases. As the balloon 150 is charged with fluid, the balloon 150 may want to inflate primarily circumferentially first. Letting number 1 indicate the initial state of inflation and number 2 indicate the state when the inflation progresses, then the circumferential stresses at state 1 and state 2 becomes;

σ_{θ1}=E_{θ1}ε_{θ1} and (2)

σ_{θ2}=E_{θ2}ε_{θ2}. (3)

When the circumferential stress (σ₀₂) becomes twice the longitudinal stress of initial state (σ_{z1}), then the longitudinal displacement (inflation) starts to take place. In this state, displacement (inflation) occurs in both the circumferential and longitudinal directions by balancing the stress state by itself. This may allow a natural inflation of the balloon determined by the dimensions of and the material properties of the balloon.

The balloon 150 may also be pre-stretched in the radial direction by attaching the balloon over the cylindrical posts 165 and 166. The ratio of the diameter of the post 165 to the inner diameter of the balloon 150 may be for example in a range of 1.2:1 to 1.6:1, which can generate 20%-60% pre-stretching of the balloon's 150 natural circumferential shape.

The ratio of the outside diameter of the balloon 150 to the balloon 150 length (or the clamp 145 to clamp 145 distance) may be determined by the shape of the infusion pump 105 and the required volume, which may vary from a range of 1:4 to 1:8. For example, in a 100 ml infusion pump 105, if the outside diameter of the balloon 150 is 0.3125 inches, then the clamp 145 to clamp 145 distance would be 1.875 inches, resulting in a ratio of 1:6. When the balloon 150 is fully inflated with a nominal volume of solution, the strain in the radial direction and the strain in the axial direction may be expected to be approximately 400% and 200%, respectively (a ratio of 2:1).

Elastomeric materials typically have an initial region in which stress increases rapidly with respect to strain increases. Beyond this region, elastomers may demonstrate a plateau region of stress until the stress ramps up rapidly again towards the end of the stress-strain curve. It may be appreciated that this steep initial region can be by-passed leading straight into the plateau region by disposing the balloon 150 pre-stretched between the end caps 115, thus relieving longitudinal stress and accelerating inflation longitudinally. This may provide a more constant flow delivery of a drug over the course of infusion as well as reduce the detrimental effects of filling pressure on the balloon's 150 structural integrity. The effect of pre-stretching may also provide tensional force enabling simultaneous contraction of the housing portions 110 and 120 toward one another to their default position. Moreover, by providing a structure encouraging longitudinal inflation, the displacement of balloon may be more linear, this allows for example, a volume indicator of remaining fluid to register closer to a linear pattern. A simple linear graduation mark now can accurately indicate the remaining volume so that a patient or caretaker can easily estimate the administration schedule.

It should be understood, of course, that the foregoing relates to exemplary embodiments of the invention and that modifications may be made without departing from the spirit and scope of the invention as set forth in the following claims.

I claim:

1. An infusion pump system, comprising:
a first housing portion including a first end cap on a sealed end of the first housing portion;
a second housing portion disposed to slide axially within the first housing portion, wherein the second housing portion includes a second end cap on a sealed end of the second housing portion, the sealed end of the second housing portion being external of the first housing portion; and
an elastomeric balloon coupled to an inlet of the first end cap and coupled to the second end cap wherein the balloon is positioned within the first and second housing portions disposed to telescope the first housing portion axially over the second housing portion when the balloon is infused with a fluid.

2. The infusion pump of claim 1, wherein the balloon includes a radial diameter configured to not contact an inner diameter of the first housing portion and an inner diameter of the second housing portion when the balloon is fully inflated with the fluid.

3. The infusion pump of claim 1, wherein the balloon is disposed pre-stretched between the first end cap and the second end cap in a default uninflated state.

4. The infusion pump of claim 1, further comprising a sealing ring adjacent an open end of the first housing portion, wherein the open end of the first housing portion is distal from the sealed end of the first housing portion.

5. An infusion pump, comprising:
a first cylindrical housing including an inner diameter;
a second cylindrical housing including an outer diameter that is less than the inner diameter of the first cylindrical housing, the second cylindrical housing being disposed to slide freely within the first cylindrical housing; and
a first end cap on the first cylindrical housing including an inlet disposed to provide fluid flow into or out of the first cylindrical housing;
wherein the second end cap on the second cylindrical housing is external of the first cylindrical housing.

6. The infusion pump of claim 5, wherein the first cylindrical housing and the second cylindrical housing are configured to simultaneously move in opposite axial directions as a fluid is introduced into the infusion pump and as the fluid is infused out to a patient.

7. The infusion pump of claim 5, further comprising a sealing ring on an open end of the first cylindrical housing, wherein the open end of the first cylindrical housing is distal from the first end cap.

8. The infusion pump of claim 5, wherein the second cylindrical housing includes an open end distal from the second end cap, the open end being disposed inside of the first cylindrical housing.

9. The infusion pump of claim 8, wherein the second cylindrical housing includes a first snap bump on an exterior wall near the open end of the second cylindrical housing and a second snap bump on an interior wall of the first cylindrical housing adjacent an open end of the first cylindrical housing, wherein the first and second snap bumps are configured to stop sliding movement of the first and second cylindrical housings at an extended position.
